(19) United States
(12) Patent Application Publication
(10) Pub. No.: US 2021/0324416 A1
(43) Pub. Date: Oct. 21, 2021

(54) VIRAL-VECTORED VACCINE FOR MALARIA

(71) Applicants: The Administrators of the Tulane Educational Fund, New Orleans, LA (US); Board of Supervisors of Louisiana State University and Agricultural and Mechanical College, Baton Rouge, LA (US)

(72) Inventors: Konstantin Kousoulas, Baton Rouge, LA (US); Paul Rider, Baton Rouge, LA (US); Ahmed Sayed Ibrahim Aly, Yalikoy Mah, Istanbul (TR)

(21) Appl. No.: 17/258,862

(22) PCT Filed: Jun. 28, 2019
(86) PCT No.: PCT/US19/39796

§ 371 Date: Jan. 8, 2021

Related U.S. Application Data
(60) Provisional application No. 62/695,307, filed on Jul. 9, 2018.

(51) Int. Cl. C12N 15/86; A61P 33/06; C07K 14/445; C07K 14/005; A61K 39/015; C12N 7/00

(52) U.S. Cl. CPC C12N 15/86; A61P 33/06; C07K 14/445; A61K 2039/6075; A61K 39/015; C12N 7/00; C07K 2319/00; C07K 14/005

(57) ABSTRACT

A malaria vaccine composition is disclosed herein that uses liver-stage parasite exported proteins as the target of a protective immune response instead of sporozoite proteins. Also disclosed is a recombinant viral particle that comprises a fusion protein disclosed herein, wherein the malaria antigen is displayed within the viral particle. Also disclosed is an isolated polynucleotide that comprises a nucleic acid sequence encoding a fusion protein disclosed herein operably linked to an expression control sequence. Also disclosed is a recombinant herpes simplex virus (HSV) genome comprising a modified VP26 gene encoding a fusion protein disclosed herein. Also disclosed is a vaccine composition that comprises a recombinant viral particle disclosed herein in a pharmaceutically acceptable excipient. In some cases, the composition further comprises an adjuvant.

### CROSS-REFERENCE TO RELATED APPLICATIONS

This application claims benefit of U.S. Provisional Application No. 62/695,307, filed Jul. 9, 2018, which is hereby incorporated herein by reference in its entirety.

### SEQUENCE LISTING

This application contains a sequence listing filed in electronic form as an ASCII.txt file entitled “222220-2150 Sequence Listing_ST25” created on Jun. 21, 2019. The content of the sequence listing is incorporated herein in its entirety.

### BACKGROUND

Malaria is one of the leading causes of global morbidity and mortality. Approximately 500 million individuals are infected with malaria each year and nearly 1 million of these individuals die from the infection. There is currently no vaccine and front-line medicines are failing due to increasing resistance. Thus, the need for a malaria vaccine is indisputable.

Considerable attention has been drawn to pre-erythrocytic stages of the malaria parasite after the seminal discoveries that immunization of mice and humans with irradiation-attenuated Plasmodium sporozoites can confer sterile protection against malaria infection. After transmission to the human host by Anopheles mosquitoes, malaria parasite sporozoites migrate from the dermis to the liver within few minutes. Once they reach the portal vein, sporozoites gain access to the liver and rapidly invade hepatocytes within a parasitophorous vacuole membrane (PVM) to initiate the asymptomatic liver stage (LS). Once inside hepatocytes, Plasmodium LS directs the export of proteins to the PVM or to the hepatocyte cytoplasm to acquire nutrients and gain control of its host cells. These parasite antigens are known as LS exported proteins. Epitopes of Plasmodium LS exported proteins are most probably displayed by MHC I complex molecules on the surface of infected hepatocytes.

Despite the evident potential of live attenuated parasite models as vaccines, the feasibility and large-scale application of live attenuated sporozoites (that have to be produced aseptically in mosquitoes in high amounts) remains difficult. To date, no other vaccine candidate has successfully conferred 100% long-term sterile protection against malaria in human volunteers like the attenuated sporozoite vaccine

### SUMMARY

A malaria vaccine composition is disclosed herein that uses liver-stage parasite exported proteins as the target of a protective immune response instead of sporozoite proteins. The sporozoite is a highly motile Plasmodium life cycle stage that is deposited in the dermis by a feeding Anopheles female mosquito. Sporozoites traverse endothelial cells in the skin to enter the blood circulation and reach the liver. Decades of efforts have concentrated heavily on neutralizing the agile sporozoites in the blood stream within this very limited window of time before it reaches the liver. Moreover, sporozoites are invasive stages that use their surface proteins as tools to traverse and invade host cells, but do not express those surface antigens anymore once they invade a replication permissive hepatocyte. Therefore, it was reasoned that vaccination with a combination of antigens that are expressed in liver stages could synergize a more potent protective immune responses against the intra-hepatocytic liver stage.

A recombinant vector was designed for vaccination with these liver stage parasite antigens. Immunization efforts over the last 40 years have shown that CD8+ cytotoxic T-cell responses are responsible for protective immunity against malaria sporozoites infections. VC2 is a recombinant herpes simplex virus type 1 containing mutations in two envelope proteins, gK and UL20 that result in the inability of VC2 to infect neurons, while it produces strong and long-lasting immune responses against herpes simplex infections. VC2 has been shown to induce strong immune responses against herpes and heterologous antigens. Measurable immunological parameters include both humoral responses (IgM, IgG, IgE) and cellular immune responses (CD4+, CD8+, etc). VC2 was selected for the production of malaria vaccines by either incorporating the malaria antigens into the capsid protein VP26, or expressed independently. As disclosed herein, this approach can be used to produce vaccines for other antigens.

Therefore, disclosed herein is a fusion protein, comprising a viral antigen fused to a heterologous viral capsid protein. In some embodiments, the antigen is a malaria protein, or an immunogenic fragment thereof, such a malaria protein selected from the group comprising EXP1, EXP2, TMP21, ICP, and UIS3.

In some embodiments, the viral capsid protein comprises HSV-1 VP26.

Also disclosed is a recombinant viral particle that comprises a fusion protein disclosed herein, wherein the malaria antigen is displayed within the viral particle.

Also disclosed is an isolated polynucleotide that comprises a nucleic acid sequence encoding a fusion protein disclosed herein operably linked to an expression control sequence.

Also disclosed is a recombinant herpes simplex virus (HSV) genome comprising a modified VP26 gene encoding a fusion protein disclosed herein.

Also disclosed is a vaccine composition that comprises a recombinant viral particle disclosed herein in a pharmaceutically acceptable excipient. In some cases, the composition further comprises an adjuvant.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows IFN-γ ELISPOTs with splenocytes from FBAC, FOVA vaccinated or unvaccinated mice (NC), demonstrating generation of ovalbumin specific T-cell responses using recombinant HSV-1.

FIGS. 2A to 2C shows construction of VC2-derived malaria vaccine. FIG. 2A illustrates design of VC2 expressing malaria antigens fused to HSV-1 capsid protein VP26. FIG. 2B shows assessment of fusion protein expression. FIG. 2C is a bar graph showing growth analysis of malaria antigen recombinant mutant.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “peptide,” “protein,” and “polypeptide” are used interchangeably to refer to a natural or synthetic molecule comprising two or more amino acids linked by the carboxyl group of one amino acid to the alpha amino group of another.

The term “protein domain” refers to a portion of a protein, portions of a protein, or an entire protein showing structural integrity; this determination may be based on amino acid composition of a portion of a protein, portions of a protein, or the entire protein.

A “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “immunogenic composition” as used herein are those which result in specific antibody production or in cellular immunity when injected into a host.

The immunogenic compositions and/or vaccines of the present disclosure may be formulated by any of the methods known in the art. They can be typically prepared as injectables or as formulations for intranasal administration, either as liquid solutions or suspensions. Solid forms suitable for solution in, or suspension in, liquid prior to injection or other administration may also be prepared. The preparation may also, for example, be emulsified, or the protein(s)/peptide(s) encapsulated in liposomes.

The active immunogenic ingredients are often mixed with excipients or carriers, which are pharmaceutically acceptable and compatible with the active ingredient. Suitable excipients include but are not limited to water, saline, dextrose, glycerol, ethanol, or the like and combinations thereof. The concentration of the immunogenic polypeptide in injectable, aerosol or nasal formulations is usually in the range of about 0.2 to 5 mg/ml. Similar dosages can be administered to other mucosal surfaces.

In addition, if desired, the vaccines may contain minor amounts of auxiliary substances such as wetting or emulsifying agents, pH buffering agents, and/or other agents, which enhance the effectiveness of the vaccine. Examples of agents which may be effective include, but are not limited to, aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE); and RIBI, which contains three components extracted from bacteria: monophosphoryl lipid A, trehalose dimycolate and cell wall skeleton (MPL+TDM+CWS) in a 2% squalene/Tween 80 emulsion. The effectiveness of the auxiliary substances may be determined by measuring the amount of antibodies (especially IgG, IgM or IgA) directed against the immunogen resulting from administration of the immunogen in vaccines which comprise the adjuvant in question. Additional formulations and modes of administration may also be used.

The immunogenic compositions and/or vaccines of the present disclosure can be administered in a manner compatible with the dosage formulation and in such amount and manner as will be prophylactically and/or therapeutically effective, according to what is known to the art. The quantity to be administered, which is generally in the range of about 1 to 1,000 micrograms of protein per dose and/or adjuvant molecule per dose, more generally in the range of about 5 to 500 micrograms of glycoprotein per dose and/or adjuvant molecule per dose, depends on the nature of the antigen and/or adjuvant molecule, subject to be treated, the capacity of the host's immune system to synthesize antibodies, and the degree of protection desired. Precise amounts of the active ingredient required to be administered may depend on the judgment of the physician or veterinarian and may be peculiar to each individual, but such a determination is within the skill of such a practitioner.

The vaccine or immunogenic composition may be given in a single dose; two-dose schedule, for example, two to eight weeks apart; or a multi-dose schedule. A multi-dose schedule is one in which a primary course of vaccination may include 1 to 10 or more separate doses, followed by other doses administered at subsequent time intervals as required to maintain and/or reinforce the immune response (e.g., at 1 to 4 months for a second dose, and if needed, a subsequent dose(s) after several months). Humans (or other animals) immunized with the virosomes of the present disclosure are protected from infection by the cognate virus.

It should also be noted that the vaccine or immunogenic composition can be used to boost the immunization of a host having been previously treated with a different vaccine such as, but not limited to, DNA vaccine and a recombinant virus vaccine.

The term “immunogenic fragment” as used herein refers to a fragment of an immunogen that includes one or more epitopes and thus can modulate an immune response or can act as an adjuvant for a co-administered antigen. Such fragments can be identified using any number of epitope mapping techniques, well known in the art (see, e.g., Epitope Mapping Protocols in Methods in Molecular Biology, Vol. 66 (Morris, G. E., Ed., 1996) Humana Press, Totowa, N.J.).

Immunogenic fragments can be at least about 2 amino acids in length, more preferably about 5 amino acids in length, and most preferably at least about 10 to about 15 amino acids in length. There is no critical upper limit to the length of the fragment, which can comprise nearly the full-length of the protein sequence or even a fusion protein comprising two or more epitopes.

The term “immunization” as used herein refers to the process of inducing a continuing protective level of antibody and/or cellular immune response which is directed against an antigen, either before or after exposure of the host to the antigen.

The term “immunogenic amount” as used herein refers to an amount capable of eliciting the production of antibodies directed against the virus in the host to which the vaccine has been administered.

The term “immunogenic carrier” as used herein refers to a composition enhancing the immunogenicity of the virosomes from any of the viruses discussed herein. Such carriers include, but are not limited to, proteins and polysaccharides, and microspheres formulated using, for example, a biodegradable polymer such as DL-lactide-coglycolide, liposomes, and bacterial cells and membranes. Protein carriers may be joined to the proteinases, or peptides derived therefrom, to form fusion proteins by recombinant or synthetic techniques or by chemical coupling. Useful carriers and ways of coupling such carriers to polypeptide antigens are known in the art.

The term “immunogenic composition” as used herein refers to a composition that comprises an antigenic molecule where administration of the composition to a subject results in the development in the subject of a humoral and/or a cellular immune response to the antigenic molecule of interest.

The term “immunological response” as used herein refers to a composition or vaccine that includes an antigen and that triggers in the host a cellular- and/or antibody-mediated immune response to antigens. Usually, such a response may include antibody production (e.g., in the intestinal tract, from germinal centers in lymph nodes, etc.), B cell proliferation, helper T cells, cytotoxic T cell proliferation, Natural Killer activation specifically to the antigen or antigens and/or fluids, secretions, tissues, cells or hosts infected therewith.

The term “immunopotentiator,” as used herein, is intended to mean a substance that, when mixed with an immunogen, elicits a greater immune response than the immunogen alone. For example, an immunopotentiator can enhance immunogenicity and provide a superior immune response. An immunopotentiator can act, for example, by enhancing the expression of co-stimulators on macrophages and other antigen-presenting cells.

The term “nucleic acid molecule” as used herein refers to DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

The terms “nucleic acid,” “nucleic acid sequence,” or “oligonucleotide” also encompass a polynucleotide. A “polynucleotide” refers to a linear chain of nucleotides connected by a phosphodiester linkage between the 3′-hydroxyl group of one nucleoside and the 5′-hydroxyl group of a second nucleoside which in turn is linked through its 3′-hydroxyl group to the 5′-hydroxyl group of a third nucleoside and so on to form a polymer comprised of nucleosides linked by a phosphodiester backbone. A “modified polynucleotide” refers to a polynucleotide in which natural nucleotides have been partially replaced with modified nucleotides.

The term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

The term “operably linked” as used herein refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

Fusion Protein

Fusion proteins, also known as chimeric proteins, are proteins created through the joining of two or more genes which originally coded for separate proteins. Translation of this fusion gene results in a single polypeptide with function properties derived from each of the original proteins. Recombinant fusion proteins can be created artificially by recombinant DNA technology for use in biological research or therapeutics. Chimeric mutant proteins occur naturally when a large-scale mutation, typically a chromosomal translocation, creates a novel coding sequence containing parts of the coding sequences from two different genes.

The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability. Thus, any of the herein disclosed functional domains can be used to design a fusion protein.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. That DNA sequence will then be expressed by a cell as a single protein. The protein can be engineered to include the full sequence of both original proteins, or only a portion of either.

If the two entities are proteins, often linker (or “spacer”) peptides are also added which make it more likely that the proteins fold independently and behave as expected. Especially in the case where the linkers enable protein purification, linkers in protein or peptide fusions are sometimes engineered with cleavage sites for proteases or chemical agents which enable the liberation of the two separate proteins. This technique is often used for identification and purification of proteins, by fusing a GST protein, FLAG peptide, or a hexa-his peptide (aka: a 6×his-tag) which can be isolated using nickel or cobalt resins (affinity chromatography). Chimeric proteins can also be manufactured with toxins or anti-bodies attached to them in order to study disease development.

Alternatively, internal ribosome entry sites (IRES) elements can be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (U.S. Pat. Nos. 5,925,565 and 5,935,819; PCT/US99/05781). IRES sequences are known in the art and include those from encephalomycarditis virus (EMCV) (Ghattas, I. R. et al., Mol. Cell. Biol., 11:5848-5849 (1991); BiP protein (Macejak and Sarnow, Nature, 353:91 (1991)); the Antennapedia gene of drosophilia (exons d and e) [Oh et al., Genes & Development, 6:1643-1653 (1992)); those in polio virus [Pelletier and Sonenberg, Nature, 334:320325 (1988); see also Mountford and Smith, TIG, 11:179-184 (1985)).

Malaria Antigens

In some embodiments, antigens of the disclosed fusion proteins is a malaria antigen. In particular, the malaria antigen can be EXP1, TMP21, or U153.

In some embodiments, the EXP1 protein has the amino acid sequence MKINIASIIFIIFSLCLVNDAYGKNKYGKNGKYGSQNVIKKHGEPVINVQDLISDMVRKE EEIVKLTKNKKSLRKINVALATALSVVSAILLGGAGLVMYNTEKGRRPFQIGKSKKGG SAMARDSSFPMNEESPLGFSPEEMEAVASKFRESMLKDGVPAPSNTPNVQN (SEQ ID NO:1), or an immunogenic fragment or variant thereof, such as NKYGKNGKYGSQNVIKKHGEPVINVQDLISDMVRKEEEIVKLTKNKKSLRKINYNTE KGRRPFQIGKSKKGGSAMARDSSFPMNEESPLGFSPEEMEAVASKFRESMLKDGV PAPSNTPNVQN (SEQ ID NO:2), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1 or 2.

As an example, an immunogenic fragment of the EXP1 protein can be encoded by the nucleic acid sequence

(SEQ ID NO: 3)
AATAAATATGGAAAGAATGGAAAATACGGTTCTCAAAACGTCATCAAGA
AGCATGGTGAGCCTGTCATCAATGTCCAAGATTTGATTTCTGACATGGT
TCGGAAGGAAGAGGAAATAGTGAAGCTGACTAAAAATAAGAAGTCTTTG
CGAAAGATAAATTACAATACAGAGAAAGGCCGGAGGCCATTCCAAATTG
GTAAGAGTAAAAAAGGCGGATCAGCAATGGCACGGGATAGCTCCTTCCC
TATGAATGAGGAATCACCCTTGGGTTTCTCTCCAGAGGAAATGGAAGCT
GTGGCATCAAAATTTCGAGAATCAATGCTTAAAGATGGCGTTCCAGCAC
CTTCCAATACTCCTAATGTACAAAAC.

In some embodiments, the TMP21 protein has the amino acid sequence MAKISKLLTFFIAFIFQASIINSLQIYLSLKPNLPKCIKERISKDTLVVGKFKTHEKESVVS IFIYDIDVNEKKINSLDKLPIFEAIDEHDIKTAFTTFYSGSYSFCAYNKSNKVVDIYFEIK HGVEARDYTKIAKADHLNEATIFLKQILNSMKTFQSNLKRIKISEEKEKKSSEKLNDTL MWFSILTIIIIIIAALTQDFYYKRFFTSKKII (SEQ ID NO:4), or an immunogenic fragment or variant thereof, such as

(SEQ ID NO: 5)
YLSLKPNLPKCIKERISKDTLVVGKFKTHEKESVVSIFIYDIDVNEKKI
NSLDKLPIFEAIDEHDIKTAFTTFYSGSYSFCAYNKSNKVVDIYFEIKH
GVEARDYTKIAKADHLNEATIFLKQILNSMKTFQSNLKRIKISEEKEKK
SSEKLNDTFYYKRFFTSKKII,

or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:4 or 5.

As an example, the TMP21 protein can be encoded by the nucleic acid sequence

(SEQ ID NO: 6)
TACTTGTCACTTAAGCCTAATTTGCCCAAATGTATAAAAGAGCGAATCA
GTAAAGATACCCTCGTGGTAGGTAAATTCAAAACACACGAAAAGGAGTC
TGTTGTTAGTATCTTCATATATGATATTGACGTTAATGAGAAAAAGATA
AATTCCCTGGATAAGTTGCCTATATTTGAGGCCATTGACGAGCACGACA
TCAAAACCGCATTCACCACCTTCTACTCTGGTAGCTACTCATTCTGTGC
TTATAACAAGTCCAATAAGGTGGTCGATATCTACTTTGAGATTAAGCAT
GGCGTAGAAGCTCGGGATTATACCAAAATTGCTAAAGCCGATCACCTGA
ATGAAGCTACCATATTCTTGAAGCAGATCCTCAATAGCATGAAAACCTT
TCAGAGCAACCTGAAAAGAATCAAAATCTCCGAGGAAAAGGAGAAGAAG
TCATCCGAGAAACTGAACGATACCTTTTATTACAAGCGGTTTTTTACTT
CCAAGAAGATTATA.

In some embodiments, the UIS3 protein has the amino acid sequence MNTLKVFFVFYVLYITTFFFNPCFCEDADYYSEIDDGALDSIDTAIKKKKKRKSVAIALL SSGLVASVIGVLYYMYKSHNKGRHDWNKGFNFFPFNKQTEYKQPDGEKPSTSTKY EEPLGVNKVNIKGKLKENNNDIDVPLKRFNTFMDNVKLAAKHHFSNLSNEQQKYLIK DYDYLRKIVQTLDENKDVNISRAQEDIAVLGVEHFLKEQYQPK (SEQ ID NO:7), or an immunogenic fragment thereof, such as YKSHNKGRHDWNKGFNFFPFNKQTEYKQPDGEKPSTSTKYEEPLGVNKVNIKGKL KENNNDIDVPLKRFNTFMDNVKLAAKHHFSNLSNEQQKYLIKDYDYLRKIVQTLDEN KDVNISRAQEDIAVLGVEHFLKEQYQPK (SEQ ID NO:8), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:7 or 8.

As an example, the UIS3 protein can be encoded by the nucleic acid sequence

(SEQ ID NO: 9)
TACAAATCCCATAACAAAGGAAGGCACGACTGGAACAAAGGTTTCAATT
TTTTCCCTTTCAACAAACAGACCGAGTACAAGCAGCCTGATGGCGAAAA
GCCCTCTACCAGTACAAAGTATGAAGAGCCTCTTGGGGTCAATAAAGTA
AACATCAAAGGGAAACTTAAAGAGAACAATAATGATATCGACGTACCAT
TGAAAAGATTCAACACCTTCATGGATAACGTGAAGCTGGCTGCAAAGCA
TCATTTTTCTAACCTGAGTAATGAACAACAAAAATACCTGATTAAAGAC
TACGACTATCTTAGGAAAATCGTACAAACTCTCGATGAGAACAAGGATG
TCAACATTAGTAGGGCTCAGGAAGACATAGCCGTTCTCGGTGTTGAACA
CTTTCTTAAAGAGCAGTACCAACCCAAA.

Viral Capsid Protein

In some embodiments, the viral capsid protein comprises HSV-1 VP26. For example, in some embodiments, the VP26 capsid protein has the amino acid sequence MAVPQFHRPSTVTTDSVRALGMRGLVLATNNSQFIMDNNHPHPQGTQGAV REFLRGQAAALTDLGLAHANNTFTPQPMFAGDAPAAWLRPAFGLRRTYSPFVVRE PSTPGTP (SEQ ID NO:10), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:10.

In some embodiments, the VP26 capsid protein can be encoded by the nucleic acid sequence

(SEQ ID NO: 11)
ATGGCCGTCCCGCAATTTCACCGCCCCAGCACCGTTACCACCGATAGCG
TCCGGGCGCTTGGCATGCGCGGGCTCGTCTTGGCCACCAATAACTCTCA
GTTTATCATGGATAACAACCACCCACACCCCCAGGGCACCCAAGGGGCC
GTGCGGGAGTTTCTCCGCGGTCAGGCGGCGGCACTGACGGACCTTGGTC
TGGCCCACGCAAACAACACGTTTACCCCGCAGCCTATGTTCGCGGGCGA
CGCACCGGCCGCCTGGTTGCGGCCCGCGTTTGGCCTGCGGCGCACCTAT
TCACCTTTTGTCGTTCGAGAACCTTCGACGCCCGGGACCCCGTGA.

Fusion Proteins

Therefore, in some embodiments the HSV VP26 Exp1 fusion protein has the amino acid sequence MAVPNKYGKNGKYGSQNVIKKHGEPVINVQDLISDMVRKEEEIVKLTKNKKSLRKIN YNTEKGRRPFQIGKSKKGGSAMARDSSFPMNEESPLGFSPEEMEAVASKFRESML KDGVPAPSNTPNVQNQFHRPSTVTTDSVRALGMRGLVLATNNSQFIMDNNHPHPQ GTQGAVREFLRGQAAALTDLGLAHANNTFTPQPMFAGDAPAAWLRPAFGLRRTYS PFVVREPSTPGTP (SEQ ID NO:12), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:12.

In some embodiments, the HSV VP26 Exp1 fusion protein can be encoded by the nucleic acid sequence

(SEQ ID NO: 13)
ATGGCCGTCCCGAATAAATATGGAAAGAATGGAAAATACGGTTCTCAAA
ACGTCATCAAGAAGCATGGTGAGCCTGTCATCAATGTCCAAGATTTGAT
TTCTGACATGGTTCGGAAGGAAGAGGAAATAGTGAAGCTGACTAAAAAT
AAGAAGTCTTTGCGAAAGATAAATTACAATACAGAGAAAGGCCGGAGGC
CATTCCAAATTGGTAAGAGTAAAAAAGGCGGATCAGCAATGGCACGGGA
TAGCTCCTTCCCTATGAATGAGGAATCACCCTTGGGTTTCTCTCCAGAG
GAAATGGAAGCTGTGGCATCAAAATTTCGAGAATCAATGCTTAAAGATG
GCGTTCCAGCACCTTCCAATACTCCTAATGTACAAAACCAATTTCACCG
CCCCAGCACCGTTACCACCGATAGCGTCCGGGCGCTTGGCATGCGCGGG
CTCGTCTTGGCCACCAATAACTCTCAGTTTATCATGGATAACAACCACC
CACACCCCCAGGGCACCCAAGGGGCCGTGCGGGAGTTTCTCCGCGGTCA
GGCGGCGGCACTGACGGACCTTGGTCTGGCCCACGCAAACAACACGTTT
ACCCCGCAGCCTATGTTCGCGGGCGACGCACCGGCCGCCTGGTTGCGGC
CCGCGTTTGGCCTGCGGCGCACCTATTCACCTTTTGTCGTTCGAGAACC
TTCGACGCCCGGGACCCCGTGA.

In some embodiments the HSV VP26 TMP21 fusion protein has the amino acid sequence MAVPYLSLKPNLPKCIKERISKDTLVVGKFKTHEKESVVSIFIYDIDVNEKKINSLDKLP IFEAIDEHDIKTAFTTFYSGSYSFCAYNKSNKVVDIYFEIKHGVEARDYTKIAKADHLN EATIFLKQILNSMKTFQSNLKRIKISEEKEKKSSEKLNDTFYYKRFFTSKKIIQFHRPST VTTDSVRALGMRGLVLATNNSQFIMDNNHPHPQGTQGAVREFLRGQAAALTDLGL AHANNTFTPQPMFAGDAPAAWLRPAFGLRRTYSPFVVREPSTPGTP (SEQ ID NO:14), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:14.

In some embodiments, the HSV VP26 TMP21 fusion protein can be encoded by the nucleic acid sequence

(SEQ ID NO: 15)
ATGGCCGTCCCGTACTTGTCACTTAAGCCTAATTTGCCCAAATGTATAA
AAGAGCGAATCAGTAAAGATACCCTCGTGGTAGGTAAATTCAAAACACA
CGAAAAGGAGTCTGTTGTTAGTATCTTCATATATGATATTGACGTTAAT
GAGAAAAAGATAAATTCCCTGGATAAGTTGCCTATATTTGAGGCCATTG
ACGAGCACGACATCAAAACCGCATTCACCACCTTCTACTCTGGTAGCTA
CTCATTCTGTGCTTATAACAAGTCCAATAAGGTGGTCGATATCTACTTT
GAGATTAAGCATGGCGTAGAAGCTCGGGATTATACCAAAATTGCTAAAG
CCGATCACCTGAATGAAGCTACCATATTCTTGAAGCAGATCCTCAATAG
CATGAAAACCTTTCAGAGCAACCTGAAAAGAATCAAAATCTCCGAGGAA
AAGGAGAAGAAGTCATCCGAGAAACTGAACGATACCTTTTATTACAAGC
GGTTTTTTACTTCCAAGAAGATTATACAATTTCACCGCCCCAGCACCGT
TACCACCGATAGCGTCCGGGCGCTTGGCATGCGCGGGCTCGTCTTGGCC
ACCAATAACTCTCAGTTTATCATGGATAACAACCACCCACACCCCCAGG
GCACCCAAGGGGCCGTGCGGGAGTTTCTCCGCGGTCAGGCGGCGGCACT
GACGGACCTTGGTCTGGCCCACGCAAACAACACGTTTACCCCGCAGCCT
ATGTTCGCGGGCGACGCACCGGCCGCCTGGTTGCGGCCCGCGTTTGGCC
TGCGGCGCACCTATTCACCTTTTGTCGTTCGAGAACCTTCGACGCCCGG
GACCCCGTGA.

In some embodiments the HSV VP26 UIS3 fusion protein has the amino acid sequence MAVPYKSHNKGRHDWNKGFNFFPFNKQTEYKQPDGEKPSTSTKYEEPLGVNKVNI KGKLKENNNDIDVPLKRFNTFMDNVKLAAKHHFSNLSNEQQKYLIKDYDYLRKIVQT LDENKDVNISRAQEDIAVLGVEHFLKEQYQPKQFHRPSTVTTDSVRALGMRGLVLAT NNSQFIMDNNHPHPQGTQGAVREFLRGQAAALTDLGLAHANNTFTPQPMFAGDAP AAWLRPAFGLRRTYSPFVVREPSTPGTP (SEQ ID NO:16), or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:16.

In some embodiments, the HSV VP26 UIS3 fusion protein can be encoded by the nucleic acid sequence

(SEQ ID NO: 17)
ATGGCCGTCCCGTACAAATCCCATAACAAAGGAAGGCACGACTGGAACA
AAGGTTTCAATTTTTTCCCTTTCAACAAACAGACCGAGTACAAGCAGCC
TGATGGCGAAAAGCCCTCTACCAGTACAAAGTATGAAGAGCCTCTTGGG
GTCAATAAAGTAAACATCAAAGGGAAACTTAAAGAGAACAATAATGATA
TCGACGTACCATTGAAAAGATTCAACACCTTCATGGATAACGTGAAGCT
GGCTGCAAAGCATCATTTTTCTAACCTGAGTAATGAACAACAAAAATAC
CTGATTAAAGACTACGACTATCTTAGGAAAATCGTACAAACTCTCGATG
AGAACAAGGATGTCAACATTAGTAGGGCTCAGGAAGACATAGCCGTTCT
CGGTGTTGAACACTTTCTTAAAGAGCAGTACCAACCCAAACAATTTCAC
CGCCCCAGCACCGTTACCACCGATAGCGTCCGGGCGCTTGGCATGCGCG
GGCTCGTCTTGGCCACCAATAACTCTCAGTTTATCATGGATAACAACCA
CCCACACCCCCAGGGCACCCAAGGGGCCGTGCGGGAGTTTCTCCGCGGT
CAGGCGGCGGCACTGACGGACCTTGGTCTGGCCCACGCAAACAACACGT
TTACCCCGCAGCCTATGTTCGCGGGCGACGCACCGGCCGCCTGGTTGCG
GCCCGCGTTTGGCCTGCGGCGCACCTATTCACCTTTTGTCGTTCGAGAA
CCTTCGACGCCCGGGACCCCGTGA.

Recombinant HSV

A recombinant HSV that can be used in the disclosed composition and methods are described in U.S. Patent Publication No. US 2017/0266275, which is incorporated by references herein for these recombinant HSV. Briefly, a recombinant HSV comprises a recombinant HSV genome, particularly a recombinant genome that is derived from the genome of a herpes simplex virus type 1 (HSV-1) or a herpes simplex virus type 2 (HSV-2). In some embodiments, the disclosed vaccines comprise attenuated, recombinant HSVs that are capable of replication in a host cell and incapable of entry into axonal compartments of neurons. For example, the recombinant HSV genomes can be engineered to comprise at least one modification in each of the UL53 and UL20 genes. The modifications in the UL53 and UL20 genes include, for example, insertions, substitutions, and deletions of one or more nucleotides that result in changes in the nucleotide sequence of each of these genes. A particular example of a recombinant HSV genome suitable for use herein is the VC2 genome. The VC2 genome, which is derived from the genome of HSV-1(F), comprises the deletion of nucleotides 112160 to 112274 from the genome of HSV-1(F), which results in the deletion of amino acids 31 to 68 in the amino terminal region of gK and the deletion of nucleotides 41339 to 41395 from the genome of HSV-1, which results in the deletion of amino acids 4-22 in the amino terminal region of the UL20 protein. A virus comprising the VC2 genome is referred to herein as “VC2” or a “VC2 virus”.

Sequences

The present compositions and methods encompass isolated or substantially purified polynucleotide (also referred to herein as “nucleic acid molecule”, “nucleic acid” and the like) or protein (also referred to herein as “polypeptide”) compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When a protein or biologically active portion thereof is recombinantly produced, optimally culture medium represents less than about 30%, 20%, 10%, 5%, or 1% (by dry weight) of chemical precursors or non-protein-of-interest chemicals.

“Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. Generally, variants of a particular gene or protein will have at least about 80%, 85%, 90%, 91%, 92%, 93%, 94%, 95%, 96%, 97%, 98%, 99% or more sequence identity to the native gene or protein as determined by sequence alignment. A biologically active variant of a protein of the invention may differ from that protein by as few as 1-15 amino acid residues, as few as 1-10, such as 6-10, as few as 5, as few as 4, 3, 2, or even 1 amino acid residue.

The disclosed proteins may be altered in various ways including amino acid substitutions, deletions, and insertions. Methods for such manipulations are generally known in the art. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

The deletions, insertions, and substitutions of the protein sequences encompassed herein are not expected to produce radical changes in the characteristics of the protein except for those changes that are disclosed herein. However, when it is difficult to predict the exact effect of the substitution, deletion, or insertion in advance of doing so, one skilled in the art will appreciate that the effect will be evaluated by routine screening assays. That is, the activity can be evaluated by assays that are disclosed hereinbelow.

Variant polynucleotides and proteins also encompass sequences and proteins derived from a mutagenic and recombinogenic procedure such as DNA shuffling. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to the polynucleotide molecules of the invention. BLAST protein searches can be performed with the XBLAST program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. LAST, Gapped BLAST, and PSI-Blast, XBLAST and NBLAST are available on the World Wide Web at ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention using BLAST with the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by BLAST using default parameters.

Pharmaceutically Acceptable Compositions

The vaccines and immunogenic compositions can further comprise one or more pharmaceutically acceptable components including, but not limited to, a carrier, an excipient, a stabilizing agent, a preservative, an immunostimulant, and an adjuvant. Each of the pharmaceutically acceptable components can be present in the vaccines and immunogenic compositions in a pharmaceutically acceptable amount. Such a pharmaceutically acceptable amount is an amount that is sufficient to produce the desired result (e.g. the amount of stabilizer sufficient to stabilize the vaccine after making and until administration) but is considered safe for administration to an animal, particularly a human.

The vaccines and other immunogenic compositions disclosed herein can comprise one or more pharmaceutically acceptable components including, but not limited to, a carrier, an excipient, a stabilizing agent, a preservative, an immunostimulant, and an adjuvant. In general, a pharmaceutically acceptable component does not itself induce the production of an immune response in the animal receiving the component and can be administered without undue toxicity in composition of the present invention.

Carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition), herein incorporated in its entirety by reference. The formulation should suit the mode of administration. In a preferred embodiment, the formulation is suitable for administration to humans, preferably is sterile, non-particulate and/or non-pyrogenic.

Examples of stabilizing agents, immunostimulants, and adjuvants include alum, incomplete Freud's adjuvant, MR-59 (Chiron), muramyl tripeptide phosphatidylethanolamide, and mono-phosphoryl Lipid A. Preservatives include, for example, thimerosal, benzyl alcohol, and parabens. Such stabilizing agents, adjuvants, immune stimulants, and preservatives are well known in the art and can be used singly or in combination.

Pharmaceutically acceptable components can include, for example, minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, and the like.

Methods of Immunization

The present invention further provides methods of immunizing a patient against an antigen comprising the step of administering to the patient a therapeutically effective amount of a vaccine comprising a fusion protein containing that antigen as disclosed herein.

In some embodiments, the therapeutically effective amount of a vaccine is administered to the subject in a single dose. In other embodiments, the vaccine is administered to the disclosed herein in multiple doses. It is recognized that the therapeutically effective amount of a vaccine can vary depending on the dosing regimen and can even vary from one administration to the next in multiple dosing regimens.

Methods of Production

Also disclosed are methods for producing a recombinant virus containing the disclosed fusion proteins. The methods comprising transfecting a host cell with the recombinant viral genome and incubating the transfected host cell under conditions favorable for the formation of a recombinant virus comprising the recombinant viral genome, whereby a recombinant virus is produced. Preferably, the host cell is an animal cell and can be either a host cell contained in an animal or an in-vitro-cultured animal cell including, for example, an in-vitro cultured human cell. The conditions under which the transfected host cell is incubated will depend on a number of factors including, but not limited to, the particular host cell, the amount of the recombinant viral genome that is transfected into the host cell, and the particular virus that is produced from the recombinant viral genome. It is recognized that those of skill in the art can determine empirically the optimal conditions for producing a recombinant virus disclosed herein in a transfected host cell by methods described elsewhere herein or otherwise known in the art. The methods can further comprise the optional step of purifying the recombinant virus by separating the recombinant virus from the cellular components of the host cell using standard methods that are known in the art.

Also disclosed are methods for producing a vaccine or immunogenic composition. The methods involve producing the recombinant virus essentially as described above. In particular, the methods for producing a vaccine or immunogenic composition comprise transfecting a host cell with the recombinant viral genome, incubating the transfected host cell under conditions favorable for the formation of a recombinant virus comprising the recombinant viral genome, purifying the recombinant virus comprising the recombinant viral genome, and optionally, combining the purified recombinant virus with at least one pharmaceutically acceptable component.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

Recombinant HSV-1 Vector Expressing Heterologous Antigen Fused to Capsid Protein is Capable of Inducing Potent and Specific T-Cell Mediated Immunity

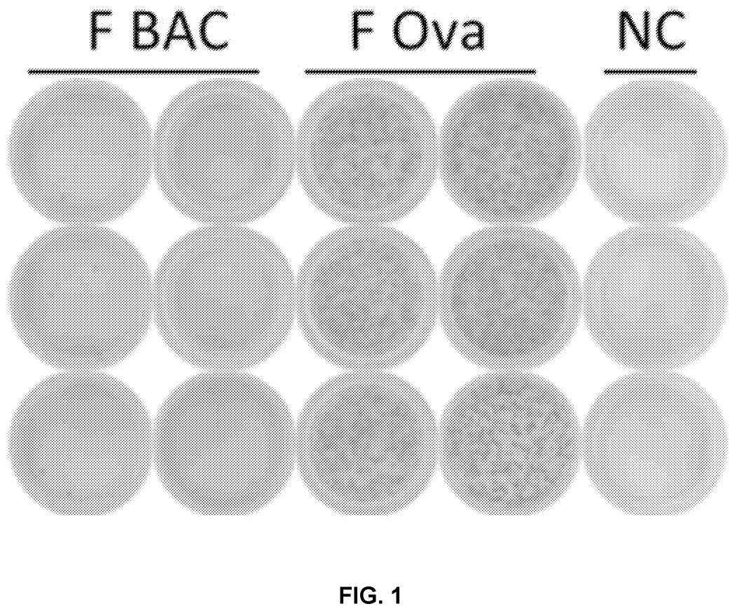

To test the ability of recombinant HSV-1 to induce cell mediated immunity to a heterologous antigen the experimental immunogen ovalbumin was fused to the minor capsid protein of HSV-1 VP26. Infectious virus possessing modified VP26 were recovered and used to vaccinate C57BI/6 mice which are of the H-2Kb haplotype and possess MHC class I capable of presenting the well-studied ovalbumin SIINFEKL (SEQ ID NO:18) epitope. Nine days post-intramuscular vaccination with either wild type (F BAC) or ovalbumin expressing virus (F Ova), mouse splenocytes were harvested, SIINFEKL (SEQ ID NO:18) peptide was added to splenocyte cultures and IFN-γ ELISPOT was performed. Splenocytes from F BAC vaccinated mice did not secrete IFN-γ when cultured with SIINFEKL (SEQ ID NO:18) peptide while splenocytes from F OVA vaccinated mice readily secreted IFN-γ when cultured with SIINFEKL (SEQ ID NO:18) peptide (FIG. 1). All vaccinated mice secreted IFN-γ when cultured with HSV-1 specific peptide and no IFN-γ when exposed to an unrelated peptide.

Construction of Recombinant VC2 Virus Expressing Malaria Antigens

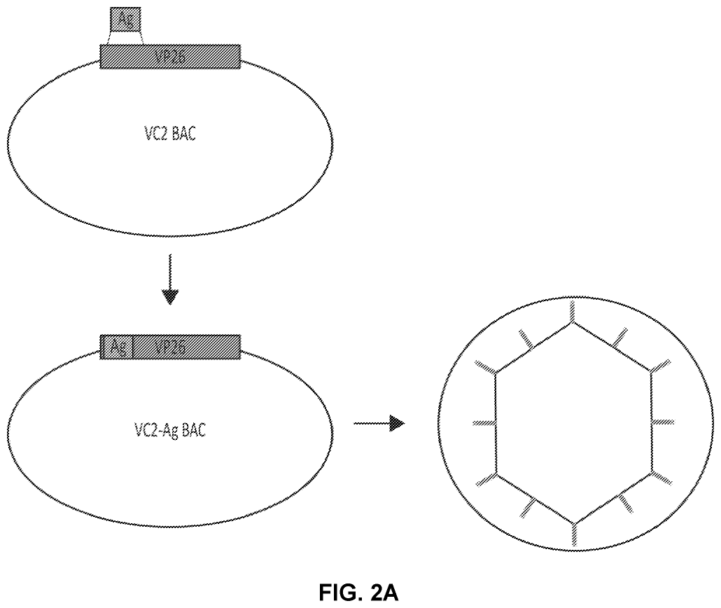

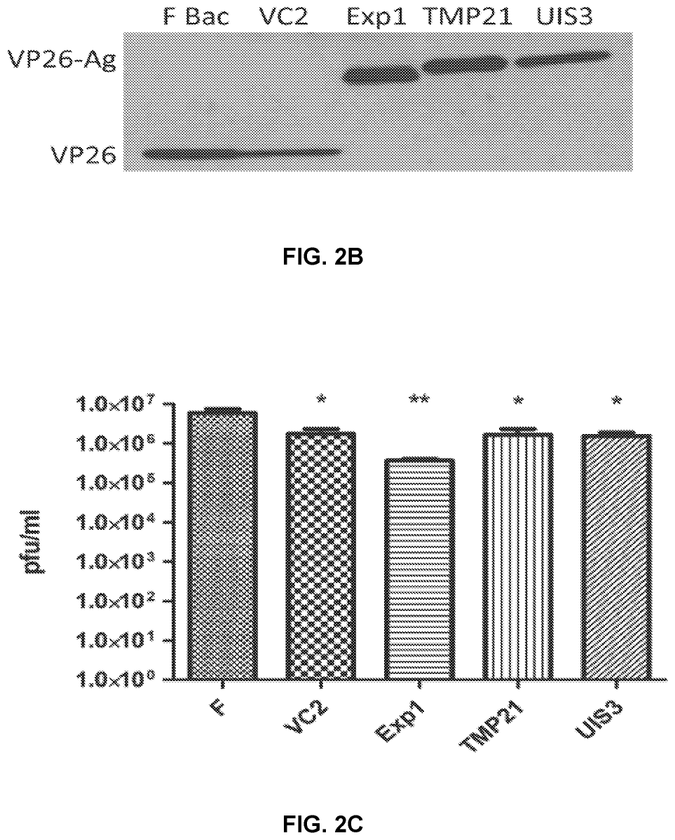

The next goal was to fuse malaria LS antigens to viral minor capsid protein VP26 (FIG. 2A). VP26 is an abundant virion protein (more than 1000 copies per virion) located in the tegument of the virion particle (between the capsid and the viral envelope). VP26 can be fused to a variety of proteins without inhibiting virion assembly and replication. Specifically, HSV1 expressing VP26 fused to the EGFP fluorescent protein has been used extensively for virus tracking experiments in vitro and in vivo without exhibiting any defects. It was hypothesized that fusion of malaria antigens to a viral protein present in the viral particle would enhance immunogenicity given that approximately 1000 copies of VP26 are present on each infectious particle. Additionally, defective viral particles present in inoculum would further increase the presence of antigen to approximately 10″11 copies in each inoculum. Finally, these viruses would be able to enter both the exogenous and endogenous antigen presentation pathways to further enhance immunogenicity. Three recombinant virus were generated, each with a different LS antigen fused to VP26: VC2-EXP1, VC2-TMP21, and VC2-UIS3. Western blots performed on protein lysates from viral stocks demonstrated the presence of VP26 fused to each malaria LS antigen (FIG. 2B). Finally growth assays were performed to determine whether fusion of LS antigens to VP26 affected the growth of these viruses. Only VC2-Exp1 exhibited growth slightly lower than parental VC2 virus (FIG. 2C).

To test the efficacy of the VC2-derived malaria vaccines, immunized mice were challenged with Plasmodium yoelii. VC2-EXP1, VC2-TMP21, and VC2-UIS3 were pooled at equal titers and administered to 6-8 week old BALB/c mice at a total dosage of 1×10⁶ plaque forming units (PFU). Mice were administered either one vaccination or a vaccination and 21-day boost. Eight weeks after final immunization mice were intravenously (IV) challenged with 500 P. yoelii salivary gland sporozoites per mouse. After challenge, parasites were detected in the peripheral blood of control mice by giemsa-stained thin blood smears (Table 1). However, in mice vaccinated with pooled VC2-derived malaria vaccines no blood stage parasites could be detected (up to 14 days following challenge), which indicates sterile protection against virulent malaria parasite sporozoite infection (Table 1).

TABLE 1
Vaccination with VC2-Malaria vaccines and subsequent
challenge with Malaria.

| | Immunization | Route of | Sterile |
|---|---|---|---|
| | Intervals | Immunization | Protected/ |
| Group (*) | in days (**) | (***) | Challenged |
| Group A: Recombinant | 1, 21 (8) | IM (500) | 8/8 |
| ExpoLS Attenuated Virus | | | |
| (8) | | | |
| Group B: Recombinant | 1, 21 (8) | IV (500) | 7/7 |
| ExpoLS Attenuated Virus | | | |
| (7) | | | |
| Group C: Recombinant | 21 (8) | IM (500) | 8/8 |
| ExpoLS Attenuated Virus | | | |
| (8) | | | |
| Group D: Recombinant | 21 (8) | IV (500) | 7/7 |
| ExpoLS Attenuated Virus | | | |
| (7) | | | |
| Group E: Control | 1, 21 (8) | IM (500) | 0/5 |
| Attenuated Virus (5) | | | |
| Group F: Control | 1, 21 (8) | IV (500) | 0/5 |
| Attenuated Virus (5) | | | |
| Group G: Naïve Control (5) | — | — | 0/5 |

(*) number of BALB/c mice per group
(**) challenge after last immunization in weeks
(***) sporozoites IV challenge dose

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A fusion protein, comprising a viral antigen fused to a heterologous viral capsid protein.

2. The fusion protein of claim 1, wherein the antigen is a malaria antigen comprising an immunogenic fragment of a protein selected from the group comprising EXP1, TMP21, and UIS3.

3. The fusion protein of claim 2, wherein the EXP1 antigen comprises the amino acid sequence SEQ ID NO:1, or an immunogenic fragment or variant thereof having at least 90% sequence identity to SEQ ID NO:1.

4. The fusion protein of claim 2, wherein the TMP21 antigen comprises the amino acid sequence SEQ ID NO:4, or an immunogenic fragment or variant thereof having at least 90% sequence identity to SEQ ID NO:4.

5. The fusion protein of claim 2, wherein the UIS3 antigen comprises the amino acid sequence SEQ ID NO:7, or an immunogenic fragment or variant thereof having at least 90% sequence identity to SEQ ID NO:7.

6. The fusion protein of claim 1, wherein the viral capsid protein comprises HSV-1 VP26.

7. The fusion protein of claim 6, wherein the HSV-1 VSP26 comprises the amino acid sequence SEQ ID NO:10, or variant thereof having at least 90% sequence identity to SEQ ID NO:10.

8. The fusion protein of claim 7, wherein the fusion protein comprises the amino acid sequence SEQ ID NO:12, SEQ ID NO:14, SEQ ID NO:16, or variant thereof having at least 90% sequence identity to SEQ ID NO:12, SEQ ID NO:14, or SEQ ID NO:16.

9. A recombinant viral particle, comprising the fusion protein of claim 1, wherein the malaria antigen is displayed on the surface of the viral particle.

10. An isolated polynucleotide, comprising a nucleic acid sequence encoding the fusion protein of claim 1 operably linked to an expression control sequence.

11. A recombinant herpes simplex virus (HSV) genome comprising a modified VP26 gene encoding the fusion protein of claim 1.

12. A vaccine composition, comprising the recombinant viral particle of claim 9 in a pharmaceutically acceptable excipient.

13. The vaccine composition of claim 12, further comprising an adjuvant.

14. A method of generating an immune response in a subject, comprising administering to the subject the vaccine composition of claim 12.


SEQUENCE LISTING (18 sequences)

SEQ ID NO: 1 - protein, 169 aa, Artificial Sequence
  Synthetic Construct
MKINIASIIFIIFSLCLVNDAYGKNKYGKNGKYGSQNVIKKHGEPVINVQDLISDMVRKEEEIVKLTKNKKSLRKINVALATALSVVSAILLGGAGLVMYNTEKGRRPFQIGKSKKGGSAMARDSSFPMNEESPLGFSPEEMEAVASKFRESMLKDGVPAPSNTPNVQN

SEQ ID NO: 2 - protein, 123 aa, Artificial Sequence
  Synthetic Construct
NKYGKNGKYGSQNVIKKHGEPVINVQDLISDMVRKEEEIVKLTKNKKSLRKINYNTEKGRRPFQIGKSKKGGSAMARDSSFPMNEESPLGFSPEEMEAVASKFRESMLKDGVPAPSNTPNVQN

SEQ ID NO: 3 - DNA, 369 bp, Artificial Sequence
  Synthetic Construct
aataaatatggaaagaatggaaaatacggttctcaaaacgtcatcaagaagcatggtgagcctgtcatcaatgtccaagatttgatttctgacatggttcggaaggaagaggaaatagtgaagctgactaaaaataagaagtctttgcgaaagataaattacaatacagagaaaggccggaggccattccaaattggtaagagtaaaaaaggcggatcagcaatggcacgggatagctccttccctatgaatgaggaatcacccttgggtttctctccagaggaaatggaagctgtggcatcaaaatttcgagaatcaatgcttaaagatggcgttccagcaccttccaatactcctaatgtacaaaac

SEQ ID NO: 4 - protein, 214 aa, Artificial Sequence
  Synthetic Construct
MAKISKLLTFFIAFIFQASIINSLQIYLSLKPNLPKCIKERISKDTLVVGKFKTHEKESVVSIFIYDIDVNEKKINSLDKLPIFEAIDEHDIKTAFTTFYSGSYSFCAYNKSNKVVDIYFEIKHGVEARDYTKIAKADHLNEATIFLKQILNSMKTFQSNLKRIKISEEKEKKSSEKLNDTLMWFSILTIIIIIIAALTQDFYYKRFFTSKKII

SEQ ID NO: 5 - protein, 168 aa, Artificial Sequence
  Synthetic Construct
YLSLKPNLPKCIKERISKDTLVVGKFKTHEKESVVSIFIYDIDVNEKKINSLDKLPIFEAIDEHDIKTAFTTFYSGSYSFCAYNKSNKVVDIYFEIKHGVEARDYTKIAKADHLNEATIFLKQILNSMKTFQSNLKRIKISEEKEKKSSEKLNDTFYYKRFFTSKKII

SEQ ID NO: 6 - DNA, 504 bp, Artificial Sequence
  Synthetic Construct
tacttgtcacttaagcctaatttgcccaaatgtataaaagagcgaatcagtaaagataccctcgtggtaggtaaattcaaaacacacgaaaaggagtctgttgttagtatcttcatatatgatattgacgttaatgagaaaaagataaattccctggataagttgcctatatttgaggccattgacgagcacgacatcaaaaccgcattcaccaccttctactctggtagctactcattctgtgcttataacaagtccaataaggtggtcgatatctactttgagattaagcatggcgtagaagctcgggattataccaaaattgctaaagccgatcacctgaatgaagctaccatattcttgaagcagatcctcaatagcatgaaaacctttcagagcaacctgaaaagaatcaaaatctccgaggaaaaggagaagaagtcatccgagaaactgaacgataccttttattacaagcggttttttacttccaagaagattata

SEQ ID NO: 7 - protein, 215 aa, Artificial Sequence
  Synthetic Construct
MNTLKVFFVFYVLYITTFFFNPCFCEDADYYSEIDDGALDSIDTAIKKKKKRKSVAIALLSSGLVASVIGVLYYMYKSHNKGRHDWNKGFNFFPFNKQTEYKQPDGEKPSTSTKYEEPLGVNKVNIKGKLKENNNDIDVPLKRFNTFMDNVKLAAKHHFSNLSNEQQKYLIKDYDYLRKIVQTLDENKDVNISRAQEDIAVLGVEHFLKEQYQPK

SEQ ID NO: 8 - protein, 140 aa, Artificial Sequence
  Synthetic Construct
YKSHNKGRHDWNKGFNFFPFNKQTEYKQPDGEKPSTSTKYEEPLGVNKVNIKGKLKENNNDIDVPLKRFNTFMDNVKLAAKHHFSNLSNEQQKYLIKDYDYLRKIVQTLDENKDVNISRAQEDIAVLGVEHFLKEQYQPK

SEQ ID NO: 9 - DNA, 420 bp, Artificial Sequence
  Synthetic Construct
tacaaatcccataacaaaggaaggcacgactggaacaaaggtttcaattttttccctttcaacaaacagaccgagtacaagcagcctgatggcgaaaagccctctaccagtacaaagtatgaagagcctcttggggtcaataaagtaaacatcaaagggaaacttaaagagaacaataatgatatcgacgtaccattgaaaagattcaacaccttcatggataacgtgaagctggctgcaaagcatcatttttctaacctgagtaatgaacaacaaaaatacctgattaaagactacgactatcttaggaaaatcgtacaaactctcgatgagaacaaggatgtcaacattagtagggctcaggaagacatagccgttctcggtgttgaacactttcttaaagagcagtaccaacccaaa

SEQ ID NO: 10 - protein, 112 aa, Artificial Sequence
  Synthetic Construct
MAVPQFHRPSTVTTDSVRALGMRGLVLATNNSQFIMDNNHPHPQGTQGAVREFLRGQAAALTDLGLAHANNTFTPQPMFAGDAPAAWLRPAFGLRRTYSPFVVREPSTPGTP

SEQ ID NO: 11 - DNA, 339 bp, Artificial Sequence
  Synthetic Construct
atggccgtcccgcaatttcaccgccccagcaccgttaccaccgatagcgtccgggcgcttggcatgcgcgggctcgtcttggccaccaataactctcagtttatcatggataacaaccacccacacccccagggcacccaaggggccgtgcgggagtttctccgcggtcaggcggcggcactgacggaccttggtctggcccacgcaaacaacacgtttaccccgcagcctatgttcgcgggcgacgcaccggccgcctggttgcggcccgcgtttggcctgcggcgcacctattcaccttttgtcgttcgagaaccttcgacgcccgggaccccgtga

SEQ ID NO: 12 - protein, 235 aa, Artificial Sequence
  Synthetic Construct
MAVPNKYGKNGKYGSQNVIKKHGEPVINVQDLISDMVRKEEEIVKLTKNKKSLRKINYNTEKGRRPFQIGKSKKGGSAMARDSSFPMNEESPLGFSPEEMEAVASKFRESMLKDGVPAPSNTPNVQNQFHRPSTVTTDSVRALGMRGLVLATNNSQFIMDNNHPHPQGTQGAVREFLRGQAAALTDLGLAHANNTFTPQPMFAGDAPAAWLRPAFGLRRTYSPFVVREPSTPGTP

SEQ ID NO: 13 - DNA, 708 bp, Artificial Sequence
  Synthetic Construct
atggccgtcccgaataaatatggaaagaatggaaaatacggttctcaaaacgtcatcaagaagcatggtgagcctgtcatcaatgtccaagatttgatttctgacatggttcggaaggaagaggaaatagtgaagctgactaaaaataagaagtctttgcgaaagataaattacaatacagagaaaggccggaggccattccaaattggtaagagtaaaaaaggcggatcagcaatggcacgggatagctccttccctatgaatgaggaatcacccttgggtttctctccagaggaaatggaagctgtggcatcaaaatttcgagaatcaatgcttaaagatggcgttccagcaccttccaatactcctaatgtacaaaaccaatttcaccgccccagcaccgttaccaccgatagcgtccgggcgcttggcatgcgcgggctcgtcttggccaccaataactctcagtttatcatggataacaaccacccacacccccagggcacccaaggggccgtgcgggagtttctccgcggtcaggcggcggcactgacggaccttggtctggcccacgcaaacaacacgtttaccccgcagcctatgttcgcgggcgacgcaccggccgcctggttgcggcccgcgtttggcctgcggcgcacctattcaccttttgtcgttcgagaaccttcgacgcccgggaccccgtga

SEQ ID NO: 14 - protein, 280 aa, Artificial Sequence
  Synthetic Construct
MAVPYLSLKPNLPKCIKERISKDTLVVGKFKTHEKESVVSIFIYDIDVNEKKINSLDKLPIFEAIDEHDIKTAFTTFYSGSYSFCAYNKSNKVVDIYFEIKHGVEARDYTKIAKADHLNEATIFLKQILNSMKTFQSNLKRIKISEEKEKKSSEKLNDTFYYKRFFTSKKIIQFHRPSTVTTDSVRALGMRGLVLATNNSQFIMDNNHPHPQGTQGAVREFLRGQAAALTDLGLAHANNTFTPQPMFAGDAPAAWLRPAFGLRRTYSPFVVREPSTPGTP

SEQ ID NO: 15 - DNA, 843 bp, Artificial Sequence
  Synthetic Construct
atggccgtcccgtacttgtcacttaagcctaatttgcccaaatgtataaaagagcgaatcagtaaagataccctcgtggtaggtaaattcaaaacacacgaaaaggagtctgttgttagtatcttcatatatgatattgacgttaatgagaaaaagataaattccctggataagttgcctatatttgaggccattgacgagcacgacatcaaaaccgcattcaccaccttctactctggtagctactcattctgtgcttataacaagtccaataaggtggtcgatatctactttgagattaagcatggcgtagaagctcgggattataccaaaattgctaaagccgatcacctgaatgaagctaccatattcttgaagcagatcctcaatagcatgaaaacctttcagagcaacctgaaaagaatcaaaatctccgaggaaaaggagaagaagtcatccgagaaactgaacgataccttttattacaagcggttttttacttccaagaagattatacaatttcaccgccccagcaccgttaccaccgatagcgtccgggcgcttggcatgcgcgggctcgtcttggccaccaataactctcagtttatcatggataacaaccacccacacccccagggcacccaaggggccgtgcgggagtttctccgcggtcaggcggcggcactgacggaccttggtctggcccacgcaaacaacacgtttaccccgcagcctatgttcgcgggcgacgcaccggccgcctggttgcggcccgcgtttggcctgcggcgcacctattcaccttttgtcgttcgagaaccttcgacgcccgggaccccgtga

SEQ ID NO: 16 - protein, 252 aa, Artificial Sequence
  Synthetic Construct
MAVPYKSHNKGRHDWNKGFNFFPFNKQTEYKQPDGEKPSTSTKYEEPLGVNKVNIKGKLKENNNDIDVPLKRFNTFMDNVKLAAKHHFSNLSNEQQKYLIKDYDYLRKIVQTLDENKDVNISRAQEDIAVLGVEHFLKEQYQPKQFHRPSTVTTDSVRALGMRGLVLATNNSQFIMDNNHPHPQGTQGAVREFLRGQAAALTDLGLAHANNTFTPQPMFAGDAPAAWLRPAFGLRRTYSPFVVREPSTPGTP

SEQ ID NO: 17 - DNA, 759 bp, Artificial Sequence
  Synthetic Construct
atggccgtcccgtacaaatcccataacaaaggaaggcacgactggaacaaaggtttcaattttttccctttcaacaaacagaccgagtacaagcagcctgatggcgaaaagccctctaccagtacaaagtatgaagagcctcttggggtcaataaagtaaacatcaaagggaaacttaaagagaacaataatgatatcgacgtaccattgaaaagattcaacaccttcatggataacgtgaagctggctgcaaagcatcatttttctaacctgagtaatgaacaacaaaaatacctgattaaagactacgactatcttaggaaaatcgtacaaactctcgatgagaacaaggatgtcaacattagtagggctcaggaagacatagccgttctcggtgttgaacactttcttaaagagcagtaccaacccaaacaatttcaccgccccagcaccgttaccaccgatagcgtccgggcgcttggcatgcgcgggctcgtcttggccaccaataactctcagtttatcatggataacaaccacccacacccccagggcacccaaggggccgtgcgggagtttctccgcggtcaggcggcggcactgacggaccttggtctggcccacgcaaacaacacgtttaccccgcagcctatgttcgcgggcgacgcaccggccgcctggttgcggcccgcgtttggcctgcggcgcacctattcaccttttgtcgttcgagaaccttcgacgcccgggaccccgtga

SEQ ID NO: 18 - protein, 8 aa, Artificial Sequence
  Synthetic Construct
SIINFEKL